(19) United States
(12) Patent Application Publication
(10) Pub. No.: US 2015/0168380 A1
(43) Pub. Date: Jun. 18, 2015

(54) MIXTURE OPHTHALMIC STRIPS

(71) Applicant: Liang Wu, Tianjin City (CN)

(72) Inventor: Liang Wu, Tianjin City (CN)

(21) Appl. No.: 14/132,233

(22) Filed: Dec. 18, 2013

(51) Int. Cl. G01N 33/50

(52) U.S. Cl. CPC G01N 33/5091

(57) ABSTRACT

This invention is within the field of eye medicine, involving the preparation method of one type of cornea intravital staining. The above mentioned staining includes the mixed solution of fluorescein sodium aqueous solution and lissamine green aqueous solution. In the above fluorescein sodium aqueous solution, the concentration of fluorescein sodium is 0.5%-4.0% w/v; while in the above lissamine green aqueous solution, the concentration of lissamine green is 0.5%-4.0% w/v. And the volume ratio of the above mentioned fluorescein sodium aqueous solution and lissamine green aqueous solution is 1:0.25-1:3. The cornea intravital staining in this invention possesses some advantages like making the staining of cornea and bulbar conjunctiva proceed at the same time, completed by one time, little irritation to eye tissue and being susceptive for patients. And it is mainly used in diagnosing and evaluating xerophthalmia, keratohelcosis, keratitis (KCS), arborized corneal epithelium herpes, and early diagnosis of Sjogren syndrome etc.

### APPLICATION FIELD

It is used to diagnose ocular disease. It is a paper strip that carries mixed dyes and stain damaged corneal and conjunctival epithelial cells on ocular surface.

### BACKGROUND

Staining method is very common in ophthalmic clinic examination to diagnose the damaged epithelial cells and connection between cells on ocular. Currently, Fluorescein Sodium, Tiger Red and Lissamine Green are common dyes used in ophthalmic clinic examination. They are dissolved into solution before using. The dye solution is very un-convenient: it has to be sterilized just before examination and spots on the skin stained by leaked dye can't be removed easily.

In order to overcome the disadvantages of the dye solution, filter paper strips carrying dye are used in diagnosing damage on ocular surface. But one strip can only carry one type of dye, upon our current knowledge.

Usually, trauma and ocular diseases cause the damage on both corneal and conjunctival epithelial cells. Different dyes are used to diagnose different damage. In order to diagnose damage on both cornea and conjunctiva, two tests have to be executed, one for corneal damage and another for conjunctival damage, because two different dyes have to be used.

Here, we successfully attach two dyes, fluoresein sodium and lissamine green to one single paper strip, which no one did before. Fluorescein sodium is usually used to diagnose damage of the epithelium cells on corneal surface, such as corneal ulcer, corneal trauma and keratitis. Tiger Red is used to diagnose dead or decayed epithelium cells on conjunctival surface. It can also stain damaged mucous layer. However, Tiger Red is irritative and weakly toxic for patient. Thus, the use of the Tiger Red is limited. Lissamine Green is similar as Tiger Red, but it is less irritative and non-toxic for patient. So this strip can be used to diagnose cornea and conjunctiva at the same time. It significantly reduces clinic testing time and also reduces patient's uncomfortable experience.

Moreover, sharp or straight tip of the strip may cause damage on ocular surface when it touches eyes. So we also cut the tip of the strip into round shape in order to reduce the damage.

### SUMMARY OF THE INVENTION

The primary object of the present invention is to use for doing multiple staining on ocular surface. This strip includes a handle made of hard paper. The end of the handle clips or sticks to a small piece of filter paper, which treated with mixed dyes, including fluorescein sodium and lissamine green.

The handle of the strip is made of a long rectangular paper, folded in the middle. The end of the handle can clip and stick to the small filter paper. And the handle of the strip can also be made of two pieces of rectangular paper, stuck together. The end of the handle can clip and stick to the small filter paper. There is a filter paper as the front tip. The shape of the filter paper is trapezoid with the narrow edge modified from straight line to round curve. For the more the mixed dyes is that such vital staining agent includes mixed solution of fluorescein sodium aqueous solution and lissamine green aqueous solution. The concentration of fluorescein sodium aqueous solution is 0.5%-4.0% w/v. The concentration of lissamine green aqueous solution is 0.5%-4.0% w/v. The volume ration of the fluorescein sodium aqueous solution and lissamine green aqueous solution is 1:0.25-1:3. And the aqueous solution refers to physiological buffer solution. The physiological buffer solution refers to borate buffer solution, phosphatic buffer solution or intraocular perfusion rinses. And how the strips are treated with shall include spraying the atomized mixed solution of fluorescein sodium aqueous solution and lissamine green aqueous solution onto paper strips, or impregnating the paper strips within the mixed solution of fluorescein sodium aqueous solution and lissamine green aqueous solution.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a view showing the top view of the Multiple-purpose strip for diagnosing damage on ocular surface.

FIG. 2 is a view showing the side view of strip according to the preferred embodiment of the present invention.

### DETAILED DESCRIPTION OF THE PREFERRED EMBODIMENTS

This invention is a multiple purpose paper strip for diagnosis of the ocular surface damage. Fluoresein sodium and lissamine green was dissolved and absorbed by strip. Strip is for single using, and cross infection is avoided. It can diagnose multiple types of damage on ocular surface. It is sterilized, safe, fast, and easy for clinic using. The problem of the leakage caused by liquid dyes can be avoided. Moreover, the round shape of the front tip can avoid damaging ocular surface if it touches eye.

A strip made of filter paper and pre-saturated by dye is a common device used to transport dye on ocular surface. But until now, no report or a tangible device used for ocular surface diagnosis indicates that more than one type of dye can be successfully attached to a single strip stably.

Usually, a trauma can cause the damage on both cornea and conjunctiva. In order to diagnose damage on both cornea and conjunctiva, two tests have to be executed, one for corneal damage and another for conjunctival damage, because two different dyes have to be used. Here, we successfully let one single strip attach two dyes, fluoresein sodium and lissamine green, so the strip can be used to diagnose cornea and conjunctiva at the same time. It significantly reduces clinic testing time and also reduces patient' s uncomfortable experience.

Liquid dyes are also used to diagnose damage on ocular surface. But liquid dye is not convenient. It needs to be sterilized just before examination, leakage of the dye can stain skin, and the spot is not easy to be removed. Our strips are pre-saturated by dyes and it is in solid status, which overcomes these disadvantages.

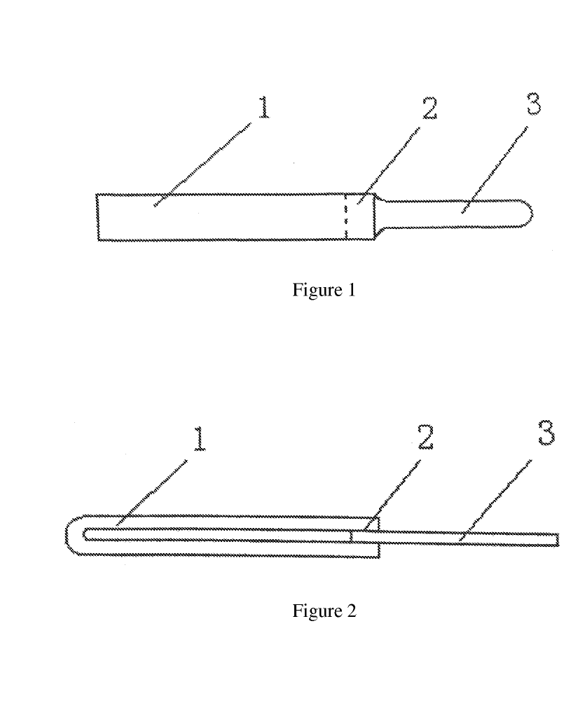

Comparing with a sharp tip or a straight tip, a round tip (FIG. 1, part 3, right side) can avoid damaging ocular surface if strip touches eyes.

Before diagnosing potential damage on ocular surface (both corneal and conjunctival cells), operator should moisten the filter paper attached to the strip by 1 or 2 drops of sterile BSS or physiological saline. Dyes absorbed by the filter paper will be dissolved by sterile BSS or physiological saline.

Dyes dissolved by sterile BSS or physiological saline will drop on ocular surface. Damaged cells on ocular surface will be stained and show different colors in a short time.

And the manufacturing method this invention include,

1. Cut a small piece of filter paper into trapezoid shape, with the narrow edge modified from straight line to round curve.
2. Cut a regular paper into long rectangular shape, with the narrow edge as the same length as the straight edge of the small filter paper. Fold the rectangular paper in the middle (part 1 in FIGS. 1 and 2). This paper will be used as a handle.
3. Prepare mixed solution of fluorescein sodium aqueous solution and lissamine green aqueous solution by dissolving fluorescein sodium and lissamine green separately with physiological buffer solution. The concentration of fluorescein sodium aqueous solution is 0.5%-4.0% w/v. The concentration of lissamine green aqueous solution is 0.5%-4.0% w/v. The volume ration of the fluorescein sodium aqueous solution and lissamine green aqueous solution is 1:0.25-1:3. Then filter the mixed solution with a filter with the diameter of 0.22 μm.
4. Spray the atomized mixed solution of fluorescein sodium aqueous solution and lissamine green aqueous solution onto paper strips (part 3 in FIGS. 1 and 2), or impregnate the paper strips (part 3 in FIGS. 1 and 2) within the mixed solution of fluorescein sodium aqueous solution and lissamine green aqueous solution.
5. Use the open side of the folded long rectangular paper to clip and stick to the small filter paper. This is the final strip as indicated by FIGS. 1 and 2.
6. The handle can also be made of two pieces of rectangular paper, stuck together.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

1. This invention is a strip used for doing multiple staining on ocular surface. This strip includes a handle made of hard paper. The end of the handle clips or sticks to a small piece of filter paper, which treated with mixed dyes, including fluorescein sodium and lissamine green.

2. As what is described in claim 1, the handle of the strip is made of a long rectangular paper, folded in the middle. The end of the handle can clip and stick to the small filter paper.

3. As what is described in claim 1, the handle of the strip can also be made of two pieces of rectangular paper, stuck together. The end of the handle can clip and stick to the small filter paper.

4. Any strips for diagnosis of the ocular surface with the properties described from claim 1, include a filter paper as the front tip. The shape of the filter paper is trapezoid with the narrow edge modified from straight line to round curve.

5. As what is described in claim 1, the mixed dyes is that such vital staining agent includes mixed solution of fluorescein sodium aqueous solution and lissamine green aqueous solution. The concentration of fluorescein sodium aqueous solution is 0.5%-4.0% w/v. The concentration of lissamine green aqueous solution is 0.5%-4.0% w/v. The volume ration of the fluorescein sodium aqueous solution and lissamine green aqueous solution is 1:0.25-1:3.

6. As what is described in claim 5, the aqueous solution refers to physiological buffer solution. The physiological buffer solution refers to borate buffer solution, phosphatic buffer solution or intraocular perfusion rinses.

7. As what is described in claim 1, how the strips are treated with shall include spraying the atomized mixed solution of fluorescein sodium aqueous solution and lissamine green aqueous solution onto paper strips, or impregnating the paper strips within the mixed solution of fluorescein sodium aqueous solution and lissamine green aqueous solution
